(19) United States
(12) Patent Application Publication
(10) Pub. No.: US 2024/0189484 A1
(43) Pub. Date: Jun. 13, 2024

(54) COMPOSITE DECELLULARIZED MATRIX MEMBRANE AND USE THEREOF

(71) Applicant: Shines Biotechnology Co., Ltd, Hangzhou (CN)

(72) Inventors: Nisi ZHANG, Hangzhou (CN); Dahong ZHANG, Hangzhou (CN); Wei WEI, Hangzhou (CN); Yi HONG, Hangzhou (CN); Shuai WANG, Hangzhou (CN); Pu ZHANG, Hangzhou (CN); Haibo TAN, Hangzhou (CN); Yidan ZHANG, Hangzhou (CN)

(21) Appl. No.: 18/177,530

(22) Filed: Mar. 2, 2023

(30) Foreign Application Priority Data
Dec. 8, 2022 (CN) 2022115694448
Dec. 8, 2022 (CN) 2022115694452
Dec. 8, 2022 (CN) 2022115694503
Dec. 8, 2022 (CN) 2022115815724

(51) Int. Cl. A61L 27/44; A61L 27/36; A61L 27/58

(52) U.S. Cl. CPC A61L 27/44; A61L 27/362; A61L 27/3629; A61L 27/3679; A61L 27/3687; A61L 27/58; A61L 2300/41; A61L 2400/02; A61L 2420/02; A61L 2430/22; A61L 2430/40

(57) ABSTRACT

The present disclosure provides a composite decellularized matrix membrane and use thereof. The composite decellularized matrix membrane prepared by the present disclosure is prepared by wrapping one layer of a polymer membrane with two layers of decellularized matrix membranes. The decellularized matrix membrane is prepared from at least a biomaterial of a porcine small intestine, a porcine bladder, and porcine skin. The polymer membrane is prepared from at least a polymer material of polycaprolactone, polydimethylsiloxane, polyurethane, polylactic acid-glycolic acid, polyvinyl alcohol, and polyhydroxy fatty acid ester. The composite decellularized matrix membrane prepared by the present disclosure can prevent calculi, makes a patient suffer less, recover faster, live better, and cost less, while medical resources are saved, and hope for treatment is also brought for some patients with a contraindication to an in-situ ileal neobladder surgery.

### CROSS-REFERENCE TO RELATED APPLICATIONS

The present application claims the priority of the prior applications in China with the Application Nos. CN202211569444.8; CN202211569445.2; CN202211569450.3; and CN202211581572.4 filed on Dec. 8, 2022. The description, claims, and drawings of the description incorporated by reference herein are incorporated in their entirety.

### TECHNICAL FIELD

The present disclosure belongs to the technical field of biomaterials and particularly relates to a composite decellularized matrix membrane for preventing calculi and use thereof.

### BACKGROUND OF THE INVENTION

An incidence of bladder cancer is the first named among urological malignancies and increases year by year. Researchers perform a sampling survey of patients who died due to malignant tumors. The result shows that the number of the dead due to bladder cancer is first ten. Bladder cancer poses a serious threat to life quality and health of patients and can be classified into muscle-invasive bladder cancer and non-muscle-invasive bladder cancer depending on whether the tumor invades a muscle layer, wherein the muscle-invasive bladder cancer is a fatal disease. Radical total cystectomy, lymph node dissection, and permanent urinary diversion are a main surgery mode for treating recurrent and multiple invasive bladder cancer at present, are also a first choice method for treating muscle-invasive bladder cancer, and can effectively reduce recurrent metastasis and improve a survival rate. However, the surgery is difficult and time-consuming, and has many complications after the surgery. The method destroys a normal structure and function of a digestive tract of a patient, also has many complications such as intestinal obstruction, metabolism disorder or nutrition disorder, mucus secretion in an artificial bladder, inflammation, calculus formation, etc. after the surgery, and seriously affects life quality of the patient after the surgery.

A non-crosslinked extracellular matrix-based biomaterial uses an animal small intestine submucosa (SIS) tissue as a raw material, such that risks such as immunogenicity and the like are removed, and a structure and active ingredients of a natural extracellular matrix are retained. After the material is implanted into a body, the material endogenously induces regeneration and repair, realizes tissue function regeneration, can be completely degraded in a body, has advantages of inflammation tolerance, adhesion prevention and the like, and is almost suitable for the regeneration and repair of all soft tissues of human body.

In order to solve the above problems of a bladder surgery, it is urgent to find a composite decellularized matrix membrane for preventing calculi.

### SUMMARY OF THE INVENTION

The present disclosure provides a composite decellularized matrix membrane for preventing calculi and use thereof. The composite decellularized matrix membrane prepared by the present disclosure is prepared by wrapping one layer of a polymer membrane with two layers of decellularized matrix membranes. The composite decellularized matrix membrane has effects on preventing water infiltration and calculi, makes a patient suffer less, recover faster, live better, and cost less, while medical resources are saved, and hope for treatment is also brought for some patients with a contraindication to an in-situ ileal neobladder surgery.

To solve the above problems, the present disclosure uses the following technical solutions.

A composite decellularized matrix membrane for preventing calculi comprises a decellularized matrix membrane and a polymer membrane. The composite decellularized matrix membrane is formed by wrapping one layer of a polymer membrane with two layers of decellularized matrix membranes.

The decellularized matrix membrane is prepared from at least a biomaterial of a porcine small intestine, a porcine bladder, and porcine skin. The polymer membrane is prepared from at least a polymer material of polycaprolactone, polylactic acid, and polyurethane.

Preferably, the decellularized matrix membrane is prepared from a porcine small intestine. Further, the polymer membrane is prepared from polycaprolactone. In some embodiments, the polymer membrane is prepared from polylactic acid. In some embodiments, the polymer membrane is prepared from polyurethane. Use of the composite decellularized matrix membrane in the preparation of an artificial bladder for preventing water infiltration. The composite decellularized matrix membrane comprises the composite decellularized matrix membrane in the above embodiment. Use of the composite decellularized matrix membrane in the preparation of an absorbably degradable artificial bladder for preventing water infiltration. The composite decellularized matrix membrane comprises the composite decellularized matrix membrane in the above embodiment. Specifically, when the decellularized matrix membrane is prepared from a porcine small intestine and the polymer membrane is polycaprolactone, polylactic acid or polyurethane, the composite decellularized matrix membrane is usable for preparing a water infiltration-preventing and/or absorbably degradable artificial bladder.

In some embodiments, the decellularized matrix membrane is prepared from at least a biomaterial of a porcine small intestine, a porcine bladder, and porcine skin. The polymer membrane is prepared from at least a polymer material of polycaprolactone, polydimethylsiloxane, polyurethane, polylactic acid-glycolic acid, polyvinyl alcohol, and polyhydroxy fatty acid ester.

Preferably, the decellularized matrix membrane is prepared from a porcine small intestine and the polymer membrane is prepared from polycaprolactone. In some embodiments, the polymer membrane is prepared from polyurethane. In some embodiments, the polymer membrane is prepared from polyvinyl alcohol. In some embodiments, the polymer membrane is prepared from polyhydroxy fatty acid ester. Use of the composite decellularized matrix membrane in the preparation of an artificial bladder for avoiding formation of calculi. The composite decellularized matrix membrane comprises the composite decellularized matrix membrane in the above embodiment. Specifically, when the polymer membrane is polycaprolactone, polydimethylsiloxane, polyurethane, polylactic acid-glycolic acid, polyvinyl alcohol, and polyhydroxy fatty acid ester, the composite decellularized matrix membrane is usable for preparing an artificial bladder for avoiding formation of calculi.

Preferably, when the decellularized matrix membrane is prepared from a porcine small intestine and the polymer membrane is polycaprolactone or polyurethane, the composite decellularized matrix membrane is usable for preparing an absorbably degradable artificial bladder for preventing calculi and water infiltration. The “preventing calculi” mentioned above refers to not producing calculi in a bladder patch, repair or bladder replacement surgery.

In some embodiments, the decellularized matrix membrane is prepared by the following steps:
(1) physically treating a biomaterial and removing an adhesive substance of a submucosa to obtain the submucosa;
(2) soaking the submucosa with a degreasing solution for 6-24 h and then rinsing the submucosa with water until there is no odor;
(3) continuously immersing the submucosa in an decellularizing solution, then placing the submucosa in a constant-temperature oscillation device for oscillating for 12-72 h, and then rinsing the submucosa with water;
(4) continuously rinsing the submucosa with salt water, placing the submucosa in a descaling solution, and then placing the submucosa in a refrigerator for 4-24 h; and
(5) rinsing the submucosa with water and freeze-drying, sealing, and storing the submucosa at 2° C.-10° C.

A method for preparing the composite decellularized matrix membrane comprises the following steps:
(1) weighing a polymer material, putting the same into a solvent, and shaking the solution until completely dissolved in a dark place at a room temperature to obtain a polymer solution;
(2) after completely soaking a decellularized matrix membrane with water, laying the membrane on a surface of a spherical mold, and placing the membrane in a ventilated place until water on a surface of the membrane is evaporated;
(3) coating the polymer solution on the surface of the decellularized matrix membrane as to obtain the decellularized matrix membrane with the polymer membrane; and
(4) after the solvent is completely volatilized, completely soaking another decellularized matrix membrane, laying the same on the decellularized matrix membrane with the polymer membrane, and after the membrane is completely dried, taking out the mold to obtain the composite decellularized matrix membrane.

The mold in step (2) is a balloon or a contractive spherical support.

A mass-volume ratio of the polymer material to the solvent in step (1) is (1-10):100. Preferably, the polymer material is selected from polycaprolactone, further, the solvent is selected from chloroform, and a preferable mass-volume ratio is 1:100.

In one aspect, the present disclosure provides a composite decellularized matrix membrane with a good toughness, wherein the composite decellularized matrix membrane comprises a decellularized matrix membrane and a polymer membrane, and the composite decellularized matrix membrane is formed by wrapping one layer of the polymer membrane with two layers of the decellularized matrix membranes.

In some embodiments, the decellularized matrix membrane is prepared from at least a biomaterial of a porcine small intestine, a porcine bladder, and porcine skin.

In some embodiments, the polymer membrane is prepared from at least a polymer material of polycaprolactone, polydimethylsiloxane, polyurethane, polylactic acid-glycolic acid, polyether-ether-ketone, and polystyrene.

In some embodiments, the decellularized matrix membrane is prepared from a porcine small intestine.

In some embodiments, the polymer membrane is prepared from polycaprolactone.

In some embodiments, the polymer membrane is prepared from polyurethane.

In some embodiments, the polymer membrane is prepared from polyether-ether-ketone.

In some embodiments, the polymer membrane is prepared from polystyrene.

In another aspect, the present disclosure provides a composite decellularized matrix membrane for preventing inflammation, wherein the composite decellularized matrix membrane comprises a decellularized matrix membrane and a polymer membrane, and the composite decellularized matrix membrane is formed by wrapping one layer of the polymer membrane with two layers of the decellularized matrix membranes.

In some embodiments, the decellularized matrix membrane is prepared from at least a biomaterial of a porcine small intestine, a porcine bladder, and porcine skin.

In some embodiments, the polymer membrane is prepared from at least a polymer material of polycaprolactone, polydimethylsiloxane, polyurethane, polylactic acid-glycolic acid, polyvinyl alcohol, and polyhydroxy fatty acid ester.

In some embodiments, the decellularized matrix membrane is prepared from a porcine small intestine.

In some embodiments, the polymer membrane is prepared from polycaprolactone.

In some embodiments, the polymer membrane is prepared from polydimethylsiloxane.

In some embodiments, the polymer membrane is prepared from polyvinyl alcohol.

In some embodiments, the polymer membrane is prepared from polyhydroxy fatty acid ester.

In some embodiments, the composite decellularized matrix membrane comprises the composite decellularized matrix membrane according to claims 1-8.

In another aspect, the present disclosure provides a composite decellularized matrix membrane for preventing deformation, wherein the composite decellularized matrix membrane comprises a decellularized matrix membrane and a polymer membrane, and the composite decellularized matrix membrane is formed by wrapping one layer of the polymer membrane with two layers of the decellularized matrix membranes, wherein the decellularized matrix membrane is prepared from at least a biomaterial of a porcine small intestine, a porcine bladder, and porcine skin.

In some embodiments, the polymer membrane is prepared from at least a polymer material of polycaprolactone, polyalkylcyanoacrylate, polylactic acid-glycolic acid, and L-polylactic acid.

In some embodiments, the decellularized matrix membrane is prepared from a porcine small intestine.

In some embodiments, the polymer membrane is prepared from polytetrafluoroethylene.

In some embodiments, the polymer membrane is prepared from polypropylene.

In some embodiments, the polymer membrane is prepared from poly(N-vinylpyrrolidone).

In some embodiments, the polymer membrane is prepared from polyethylene or polyvinylpyrrolidone.

In some embodiments, the present disclosure provides use of the composite decellularized matrix membrane in the preparation of a ureter for preventing inflammation. The composite decellularized matrix membrane comprises the aforementioned composite decellularized matrix membrane. The polymer membrane is prepared from at least a polymer material of polycaprolactone, polyalkylcyanoacrylate, polylactic acid-glycolic acid, and L-polylactic acid.

The present disclosure has the following beneficial effects:
1. The decellularized matrix membrane of the present disclosure is prepared from a porcine small intestine, a polymer material of polycaprolactone is compounded to the decellularized matrix membrane to construct a membrane material, such that a mechanical strength of the decellularized porcine small intestine is improved, and the membrane also has a function of preventing water infiltration. This is to solve problems that the decellularized matrix membrane is softened and deformed with water and accompanied by water infiltration by using characteristics that the polymer material is waterproof and easily adhered to a surface of an object, and has good biocompatibility.
2. The source material of the present disclosure is easy to obtain, simple to prepare, and easy for a mass production, and has a low cost.
3. A patient subjected to a bladder surgery using the composite decellularized matrix membrane does not need an abdominal wall stoma, an appearance of the patient is not influenced, and the patient does not need to wear a urine collection bag for the whole life like a patient with an ectopic urinary diversion. In addition, the present disclosure has advantages of less complications and no damage to the intestinal tract. The patient is not like a patient subjected to a sigmoid colorectal cystectomy who easily suffers from serious complications such as urinary tract inflammation, water electrolyte acid-base equilibrium disorder, even intestinal tumors and the like. Compared with an in-situ ileal neobladder surgery, the technology does not need an intestinal tract surgery, reduces a surgical difficulty, shortens surgical time, and reduces a surgical cost. After a tissue is regenerated, an artificial bladder body material is degraded, absorbed, and free of residue. A healed bladder is mainly an autologous tissue. Therefore, the technology makes a patient suffer less, recover faster, live better, and cost less, at the same time, saves medical resources, and can also bring hope for treatment for some patients with a contraindication to an in-situ ileal neobladder surgery.
4. A spherical artificial bladder manufactured according to a mass-volume ratio of the polymer material to a solvent provided by the present disclosure has advantages of quick dissolution, good fluidity, and quick drying. When the composite decellularized matrix membrane prepared by the present disclosure is used for an artificial bladder, no calculus is formed, which indicates that the artificial bladder prepared from the composite decellularized matrix membrane provided by the present disclosure in clinical practice does not enable a patient to be like a patient subjected to a sigmoid colorectal cystectomy who easily suffers from serious complications such as urinary tract inflammation, water electrolyte acid-base equilibrium disorder, even intestinal tumors and the like. The artificial bladder has few complications, does not damage an intestinal tract, and greatly reduces surgical risks and patient injury.

### BRIEF DESCRIPTION OF DRAWINGS

FIG. 1 shows the decellularized matrix membrane provided in example 1.1;

FIG. 2 shows the composite decellularized matrix membrane provided in example 5.1;

FIG. 3 is a flowchart of a preparation method provided by the present disclosure;

FIG. 4 shows a water infiltration-preventing experiment;

FIG. 5 shows suture in an absorbable degradation experiment 1 month after a rabbit bladder surgery;

FIG. 6 shows a state of an experimental rabbit in an absorbable degradation experiment 1 month after a rabbit bladder surgery;

FIG. 7 shows a first pathological figure of an experimental rabbit in an absorbable degradation experiment;

FIG. 8 shows a second pathological figure of an experimental rabbit in an absorbable degradation experiment;

FIG. 9 shows a surgical condition of an experimental pig in an absorbable degradation experiment;

FIG. 10 shows first pathological sections of an experimental pig in an absorbable degradation experiment 2 months after a surgery;

FIG. 11 shows second pathological sections of an experimental pig in an absorbable degradation experiment 2 months after a surgery;

FIG. 12 shows a surgery of example 5.1 in an anti-inflammatory animal experiment of a bladder;

FIG. 13 shows a surgery of example 8 in an anti-inflammatory animal experiment of a bladder;

FIG. 14 shows a surgery of example 11 in an anti-inflammatory animal experiment of a bladder;

FIG. 15 shows a surgery of example 15 in an anti-inflammatory animal experiment of a bladder;

FIG. 16 shows an experimental result in a calculus-preventing experiment;

FIG. 17 shows a test result of example 5.1 in a toughness experiment;

FIG. 18 shows a test result of example 8 in a toughness experiment;

FIG. 19 shows a test result of example 11 in a toughness experiment;

FIG. 20 shows a test result of example 13 in a toughness experiment;

FIG. 21 shows a test result of example 12 in a toughness experiment;

FIG. 22 shows a test result of example 15 in a toughness experiment;

FIG. 23 shows a surgical condition of example 17 in an anti-inflammatory animal experiment of a ureter;

FIG. 24 shows a surgical condition of example 19 in an anti-inflammatory animal experiment of a ureter;

FIG. 25 shows a surgical condition of example 23 in an anti-inflammatory animal experiment of a ureter;

FIG. 26 shows a surgical condition of example 17 in an anti-inflammatory animal experiment of a ureter;

FIG. 27 shows a surgical condition of example 21 in an anti-inflammatory animal experiment of a ureter;

FIG. 28 shows a cystography of a urethral catheter in example 17 in an anti-inflammatory animal experiment of a ureter 1 week after a surgery;

FIG. 29 shows a test device for a rigidity and strength experiment;

FIG. 30 is a schematic diagram of a test for a rigidity and strength experiment;

FIG. 31 is a schematic structural diagram of the composite decellularized matrix membrane according to the present disclosure;

FIG. 32 is a schematic diagram of the spherical composite decellularized matrix membrane prepared in example 6;

FIG. 33 is a schematic diagram of the tubular composite decellularized matrix membrane prepared in example 17;

FIG. 34 shows a fluorescence intensity of experiment 1 in an anti-inflammatory cell experimental of a bladder; and

FIG. 35 shows a cell viability of experiment 2 in an anti-inflammatory cell experimental of a bladder.

### DETAILED DESCRIPTION OF THE INVENTION

In order to describe the present disclosure more specifically, the technical solutions of the present disclosure will be described in detail below in combination with the drawings and specific embodiments. These descriptions only show how the present disclosure is realized and do not limit the specific scope of the present disclosure. The scope of the present disclosure is defined by the claims.

### Example 1.1 Preparation of Decellularized Matrix Membrane (Biomaterial-Porcine Small Intestine)

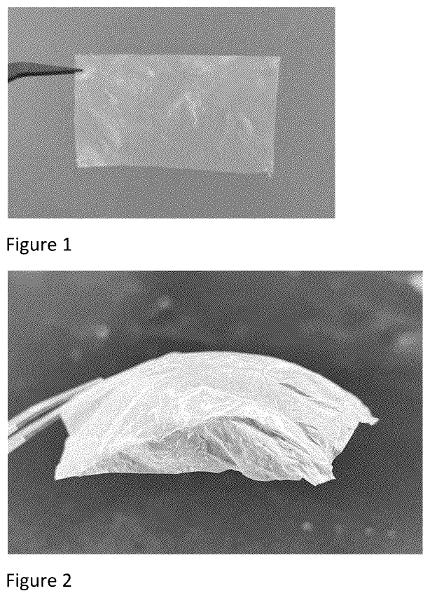

The decellularized matrix membrane provided by the present example comprised the following preparation steps:
(1) a porcine small intestine was physically treated and an adhesive substance of a submucosa was removed to obtain the submucosa;
(2) the obtained submucosa of the porcine small intestine was immersed in a decellularizing solution (10 ml of Triton and 1 ml of ammonium hydroxide was added to 1,000 mL of deionized water) and the submucosa was placed in a refrigerator at 4° C. for oscillating at 100 rpm for 12 h;
(3) after rinsing with a large amount of deionized water, placing the submucosa in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine small intestine; and
(5) finally the obtained decellularized matrix membrane was repeatedly washed with deionized water, freeze-dried, sealed, and stored in a refrigerator at 4° C. The prepared decellularized matrix membrane in the example was shown in FIG. 1.

### Example 1.2 Preparation of Decellularized Matrix Membrane (Biomaterial-Porcine Bladder)

The decellularized matrix membrane provided by the present example comprised the following preparation steps:
(1) a porcine bladder was physically treated and an adhesive substance of a submucosa was removed to obtain the submucosa;
(2) the obtained submucosa of the porcine bladder was immersed in a decellularizing solution (10 mL of Triton and 1 ml of ammonium hydroxide was added to 1,000 mL of deionized water) and the submucosa was placed in a refrigerator at 4° C. for oscillating at 100 rpm for 12 h;
(3) after rinsing with a large amount of deionized water, the submucosa was placed in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine bladder; and
(5) finally the obtained decellularized matrix membrane was repeatedly washed with deionized water, freeze-dried, sealed, and stored in a refrigerator at 4° C.

### Example 1.3 Preparation of Decellularized Matrix Membrane (Biomaterial-Porcine Skin)

The decellularized matrix membrane provided by the present example comprised the following preparation steps:
(1) porcine skin was physically treated and an adhesive substance of a submucosa was removed to obtain the submucosa;
(2) the obtained submucosa of the porcine skin was immersed in a decellularizing solution (10 mL of Triton and 1 mL of ammonium hydroxide was added to 1,000 mL of deionized water) and the submucosa was placed in a refrigerator at 4° C. for oscillating at 100 rpm for 12 h;
(3) after rinsing with a large amount of deionized water, the submucosa was placed in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine skin; and
(5) finally the obtained decellularized matrix membrane was repeatedly washed with deionized water, freeze-dried, sealed, and stored in a refrigerator at 4° C.

### Example 1.4 Preparation of Decellularized Matrix Membrane (Biomaterial-Porcine Small Intestine and Porcine Bladder)

The decellularized matrix membrane provided by the present example comprised the following preparation steps:
(1) a porcine small intestine and a porcine bladder were physically treated and an adhesive substance of a submucosa was removed to obtain the submucosa;
(2) the obtained submucosa of the porcine small intestine and the porcine bladder was immersed in a decellularizing solution (10 mL of Triton and 1 mL of ammonium hydroxide was added to 1,000 mL of deionized water) and the submucosa was placed in a refrigerator at 4° C. for oscillating at 100 rpm for 12 h;
(3) after rinsing with a large amount of deionized water, the submucosa was placed in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine small intestine and the porcine bladder; and
(5) finally the obtained decellularized matrix membrane was repeatedly washed with deionized water, freeze-dried, sealed, and stored in a refrigerator at 4° C.

### Example 1.5 Preparation of Decellularized Matrix Membrane (Biomaterial-Porcine Small Intestine and Porcine Skin)

The decellularized matrix membrane provided by the present example comprised the following preparation steps:
(1) a porcine small intestine and porcine skin were physically treated and an adhesive substance of a submucosa was respectively removed to obtain the submucosa;
(2) the obtained submucosa of the porcine small intestine and the porcine skin was immersed in a decellularizing solution (10 mL of Triton and 1 mL of ammonium hydroxide was added to 1,000 mL of deionized water) and the submucosa was placed in a refrigerator at 4° C. for oscillating at 100 rpm for 12 h;
(3) after rinsing with a large amount of deionized water, the submucosa was placed in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine small intestine and the porcine skin; and
(5) finally the obtained decellularized matrix membrane was repeatedly washed with deionized water, freeze-dried, sealed, and stored in a refrigerator at 4° C.

### Example 1.6 Preparation of Decellularized Matrix Membrane (Biomaterial-Porcine Bladder and Porcine Skin)

The decellularized matrix membrane provided by the present example comprised the following preparation steps:
(1) a porcine bladder and porcine skin were physically treated and an adhesive substance of a submucosa was respectively removed to obtain the submucosa;
(2) the obtained submucosa of the porcine bladder and the porcine skin was immersed in a decellularizing solution (10 mL of Triton and 1 mL of ammonium hydroxide was added to 1,000 mL of deionized water) and the submucosa was placed in a refrigerator at 4° C. for oscillating at 100 rpm for 12 h;
(3) after rinsing with a large amount of deionized water, the submucosa was placed in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine bladder and the porcine skin; and
(5) finally the obtained decellularized matrix membrane was repeatedly washed with deionized water, freeze-dried, sealed, and stored in a refrigerator at 4° C.

### Example 1.7 Preparation of Decellularized Matrix Membrane (Biomaterial-Porcine Small Intestine, Porcine Bladder, and Porcine Skin)

The decellularized matrix membrane provided by the present example comprised the following preparation steps:
(1) a porcine small intestine, a porcine bladder and porcine skin were physically treated and an adhesive substance of a submucosa was respectively removed to obtain the submucosa;
(2) the obtained submucosa of the porcine small intestine, the porcine bladder, and the porcine skin was immersed in a decellularizing solution (10 mL of Triton and 1 mL of ammonium hydroxide was added to 1,000 mL of deionized water) and the submucosa was placed in a refrigerator at 4° C. for oscillating at 100 rpm for 12 h;
(3) after rinsing with a large amount of deionized water, the submucosa was placed in 1 L of a degreasing solution (methanol/chloroform=1:1) for soaking for 24 h;
(4) after rinsing with deionized water, the submucosa was placed in 1 L of anhydrous ethanol for oscillating and soaking for 24 h and residual methanol/chloroform was fully extracted to obtain a prepared decellularized matrix membrane of the submucosa of the porcine small intestine, the porcine bladder, and the porcine skin; and
(5) finally the obtained decellularized matrix membrane was repeatedly washed with deionized water, freeze-dried, sealed, and stored in a refrigerator at 4° C.

### Example 2 Preparation of Decellularized Matrix Membrane (Biomaterial-Porcine Small Intestine and Porcine Skin)

The decellularized matrix membrane provided by the present example comprised the following preparation steps:
(1) a porcine small intestine and porcine skin were physically treated and an adhesive substance of a submucosa was removed to obtain the submucosa of the porcine small intestine and the porcine skin;
(2) the obtained submucosa of the porcine small intestine and the porcine skin was immersed in a decellularizing solution (0.05 g trypsin, 0.05 g ethylenediaminetetraacetic acid disodium, and 100 ml water) and the submucosa was placed in a constant-temperature oscillation device for oscillating at 37° C. and 100 rpm for 12 h;
(3) after rinsing with deionized water, the submucosa was placed in a decellularizing solution (10 mL of Triton and 1 mL of ammonium hydroxide was added to 1,000 mL of deionized water) and the submucosa was placed in a refrigerator at 4° C. for soaking for 24 h;
(4) after rinsing with deionized water, the submucosa was placed 1-2 L of a degreasing solution (methanol/chloroform=1/1) for soaking for 24 h;
(5) after rinsing with deionized water, the submucosa was placed in 1-2 L of anhydrous ethanol for oscillating and soaking at 100 rpm for 24 h and residual methanol/chloroform was fully extracted to obtain a prepared decellularized matrix membrane; and
(6) finally the obtained decellularized matrix membrane was repeatedly washed with deionized water, freeze-dried, sealed, and stored in a refrigerator at 4° C.

### Example 3 Preparation of Decellularized Matrix Membrane (Biomaterial-Porcine Skin and Porcine Bladder)

The decellularized matrix membrane provided by the present example comprised the following preparation steps:
(1) a biomaterial was physically treated and an adhesive substance of a submucosa was removed to obtain the submucosa of porcine skin and a porcine bladder;
(2) the submucosa of the porcine skin and the porcine bladder was soaked with 1-2 L of a degreasing solution (methanol/chloroform=1/1) for 24 h and then rinsed with deionized water for 5 times until there is no obvious odor;
(3) the obtained submucosa of the porcine skin and the porcine bladder was immersed in a decellularizing solution (0.05 g trypsin, 0.05 g ethylenediaminetetraacetic acid disodium, and 100 mL water) and the submucosa was placed in a constant-temperature oscillation device for oscillating at 37° C. and 100 rpm for 12 h and then rinsed with deionized water;
(4) after rinsing with normal saline for 3 times, the submucosa was placed in a descaling solution (5 g sodium dodecyl sulfate and 1,000 mL water) and in a refrigerator at 4° C. for 24 h; and
(5) finally the obtained decellularized matrix membrane was repeatedly washed with deionized water, drained, freeze-dried, sealed, and stored in a refrigerator at 4° C.

### Example 4 Preparation of Decellularized Matrix Membrane (Biomaterial-Porcine Small Intestine, Porcine Skin, and Porcine Bladder)

The decellularized matrix membrane provided by the present example comprised the following preparation steps:
(1) a biomaterial was physically treated and an adhesive substance of a submucosa was removed to obtain the submucosa of a porcine small intestine, porcine skin, and a porcine bladder;
(2) the submucosa was soaked with 1-2 L of a degreasing solution (methanol/chloroform=1/1) for 24 h, mixed, and then rinsed with deionized water for 5 times until there is no obvious odor;
(3) the obtained mixed submucosa was immersed in 1,000 mL of a decellularizing solution (0.05 g trypsin, 0.05 g ethylenediaminetetraacetic acid disodium, and 100 mL water) and the submucosa was placed in a constant-temperature oscillation device for oscillating at 37° C. and 100 rpm for 12 h and then rinsed with deionized water;
(4) after rinsing with deionized water for 3 times, the submucosa was placed in a descaling solution (10 mL of Triton and 1 mL of ammonium hydroxide was added to 1,000 mL of deionized water) and the submucosa was placed in a refrigerator at 4° C. for soaking for 24 h; and
(5) finally the obtained decellularized matrix membrane was rinsed with deionized water 4 times, drained, freeze-dried, sealed, and stored in a refrigerator at 4° C.

### Example 5.1 Preparation of Composite Decellularized Matrix Membrane (PCL Polymer Membrane-Porcine Small Intestine)

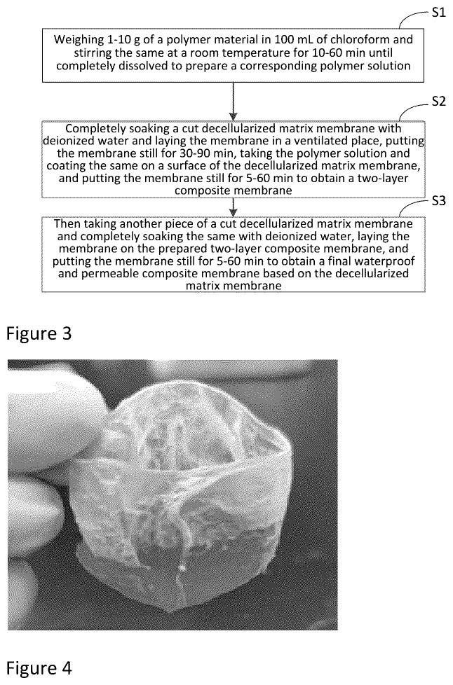

The composite decellularized matrix membrane provided by the present example comprised the following preparation steps as shown in FIG. 3:
(1) 1 g of polycaprolactone (PCL) was weighed and added to 100 mL of chloroform, and the mixture was wrapped in tin paper to avoid light and stirred at a room temperature for 60 min until completely dissolved to obtain a polymer solution;
(2) a piece of a 4 cm×4 cm decellularized matrix membrane of example 1.1 was taken, completely soaked with deionized water, laid on the cut decellularized matrix membrane, and put still in a ventilated place for 60 min; after water on a surface was evaporated, 2 mL of the polymer solution was taken and coated on a surface of the decellularized matrix membrane, and the decellularized matrix membrane was put still for 60 min after a solvent chloroform was completely volatilized to obtain the decellularized matrix membrane with the polymer material; and
(3) then a piece of 4 cm×4 cm decellularized matrix membrane of example 1.1 was completely soaked with deionized water, laid on the prepared decellularized matrix membrane with the polymer material, and put still for 60 min to obtain a final composite decellularized matrix membrane after completely air-dried as shown in FIG. 2 and FIG. 31.

### Example 5.2 Preparation of Composite Decellularized Matrix Membrane (PCL Polymer Membrane-Porcine Bladder)

The composite decellularized matrix membrane provided by the present example comprised the following preparation steps:
(1) 1 g of polycaprolactone (PCL) was weighed and added to 100 mL of chloroform, and the mixture was wrapped in tin paper to avoid light and stirred at a room temperature for 60 min until completely dissolved to obtain a polymer solution;
(2) a piece of a 4 cm×4 cm decellularized matrix membrane of example 1.2 was taken, completely soaked with deionized water, laid on the cut decellularized matrix membrane, and put still in a ventilated place for 60 min; after water on a surface was evaporated, 2 mL of the polymer solution was taken and coated on a surface of the decellularized matrix membrane, and the decellularized matrix membrane was put still for 60 min after a solvent chloroform was completely volatilized to obtain the decellularized matrix membrane with the polymer material; and
(3) then a piece of 4 cm×4 cm decellularized matrix membrane of example 1.2 was completely soaked with deionized water, laid on the prepared decellularized matrix membrane with the polymer material, and put still for 60 min to obtain a final composite decellularized matrix membrane after completely air-dried.

### Example 5.3 Preparation of Composite Decellularized Matrix Membrane (PCL Polymer Membrane-Porcine Skin)

The composite decellularized matrix membrane provided by the present example comprised the following preparation steps:
(1) 1 g of polycaprolactone (PCL) was weighed and added to 100 mL of chloroform, and the mixture was wrapped in tin paper to avoid light and stirred at a room temperature for 60 min until completely dissolved to obtain a polymer solution;
(2) a piece of a 4 cm×4 cm decellularized matrix membrane of example 1.3 was taken, completely soaked with deionized water, laid on the cut decellularized matrix membrane, and put still in a ventilated place for 60 min; after water on a surface was evaporated, 2 mL of the polymer solution was taken and coated on a surface of the decellularized matrix membrane, and the decellularized matrix membrane was put still for 60 min after a solvent chloroform was completely volatilized to obtain the decellularized matrix membrane with the polymer material; and
(3) then a piece of 4 cm×4 cm decellularized matrix membrane of example 1.3 was completely soaked with deionized water, laid on the prepared decellularized matrix membrane with the polymer material, and put still for 60 min to obtain a final composite decellularized matrix membrane after completely air-dried.

### Example 5.4 Preparation of Composite Decellularized Matrix Membrane (PCL Polymer Membrane-Porcine Small Intestine and Porcine Bladder)

The composite decellularized matrix membrane provided by the present example comprised the following preparation steps:
(1) 1 g of polycaprolactone (PCL) was weighed and added to 100 mL of chloroform, and the mixture was wrapped in tin paper to avoid light and stirred at a room temperature for 60 min until completely dissolved to obtain a polymer solution;
(2) a piece of a 4 cm×4 cm decellularized matrix membrane of example 1.4 was taken, completely soaked with deionized water, laid on the cut decellularized matrix membrane, and put still in a ventilated place for 60 min; after water on a surface was evaporated, 2 mL of the polymer solution was taken and coated on a surface of the decellularized matrix membrane, and the decellularized matrix membrane was put still for 60 min after a solvent chloroform was completely volatilized to obtain the decellularized matrix membrane with the polymer material; and
(3) then a piece of 4 cm×4 cm decellularized matrix membrane of example 1.4 was completely soaked with deionized water, laid on the prepared decellularized matrix membrane with the polymer material, and put still for 60 min to obtain a final composite decellularized matrix membrane after completely air-dried.

### Example 6 Preparation of Composite Waterproof Balloon—Polycaprolactone (PCL)

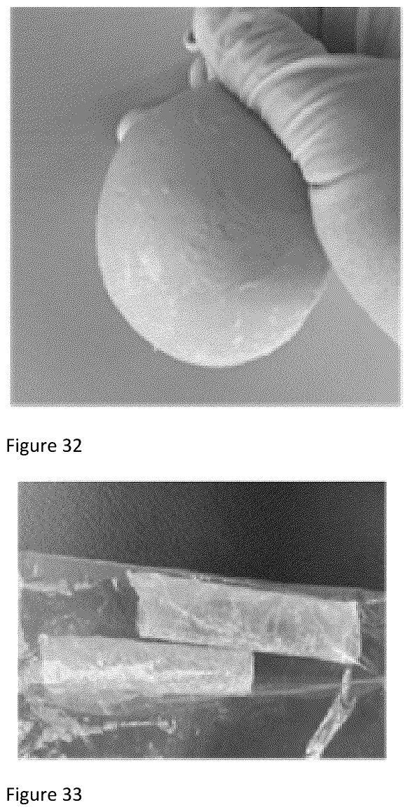

The composite waterproof balloon provided by the present example comprised the following preparation steps:
(1) preparation of PCL solution: 5 g of polycaprolactone (PCL) was weighed and added to 500 mL of chloroform, and the mixture was wrapped in tin paper to avoid light and stirred at a room temperature for 1 h until completely dissolved;
(2) 500 ml of deionized water was injected into a balloon and the balloon was sealed;
(3) the decellularized matrix membrane of example 1.1 was completely soaked, laid on a surface of the balloon, and placed in a ventilated place until water on a surface was evaporated;
(4) 10 mL of the PCL solution was taken and coated on the surface of the decellularized matrix membrane until the solvent chloroform was completely volatilized;
(5) then a piece of the completely soaked decellularized matrix membrane (example 1.1) with an appropriate size was taken and carefully laid on the decellularized matrix membrane with a polymer membrane manufactured in step (4) until the membrane was completely dried; and
(6) deionized water in the balloon was poured out and the balloon was taken out to obtain a spherical composite decellularized matrix membrane as shown in FIG. 32.

### Example 7 Preparation of Composite Waterproof Balloon—Polylactic Acid (PLA)

The composite waterproof balloon provided by the present example comprised the following preparation steps:
(1) a hollow spherical tool capable of dividing into two parts and a spherical support with a contractive hollow part were taken; and
(2) the decellularized matrix membrane in example 1.1 was soaked with deionized water and adhered to a surface of the spherical support fully, a vacuum device was connected, a vacuum degree was adjusted, bubbles in the decellularized matrix membrane were removed, the vacuum device was dismantled, after water on a surface of the membrane was evaporated, a polymer solution (a polymer material used in the example was polylactic acid and others were the same as example 6) prepared according to step (1) in example 6 was evenly coated on the surface of the decellularized matrix membrane, after chloroform was volatilized, another completely soaked decellularized matrix membrane was carefully laid, then the vacuum device was connected again, bubbles in the decellularized matrix membrane were removed, the vacuum device was dismantled, the spherical tool was combined and wrapped the spherical support to prevent a material from tilting, after the membrane was completely air-dried, and the spherical support was taken down, the contractive spherical support was taken out to obtain a spherical composite decellularized matrix membrane.

### Comparative Example 1 Preparation of Composite Waterproof Balloon—Polyalkylcyanoacrylate (PACA)

The composite waterproof balloon provided by the present example comprised the following preparation steps:
(1) preparation of PCL solution: 5 g of polyalkylcyanoacrylate (PACA) was weighed and added to 500 mL of chloroform, and the mixture was wrapped in tin paper to avoid light and stirred at a room temperature for 1 h until completely dissolved;
(2) 500 ml of deionized water was injected into a balloon and the balloon was sealed;
(3) the decellularized matrix membrane of example 1.1 was completely soaked, laid on a surface of the balloon, and placed in a ventilated place until water on a surface was evaporated; (4) 10 mL of the PACA solution was taken and coated on the surface of the decellularized matrix membrane until the solvent chloroform was completely volatilized;
(5) then a piece of the completely soaked decellularized matrix membrane (example 1.1) with an appropriate size was taken and carefully laid on the decellularized matrix membrane with a polymer membrane manufactured in step (4) until the membrane was completely dried; and
(6) deionized water in the balloon was poured out and the balloon was taken out to obtain a spherical composite decellularized matrix membrane.

### Comparative Example 2 Preparation of Composite Waterproof Balloon—L-Polylactic Acid (PLLA)

The preparation steps of the present comparative example were the same as those of comparative example 1. A difference was that the polymer material is L-polylactic acid.

### Water Infiltration-Preventing Performance Test

Test samples: spherical composite decellularized matrix membranes prepared in examples 6 and 7 and comparative examples 1 and 2;

Test method: a non-testing end was blocked, artificial urine at a temperature of 37+/−2° C. was filled into the spherical composite decellularized matrix membrane, the spherical composite decellularized matrix membrane was placed at a room temperature for 4 h, whether liquid seeps or not on a surface of the spherical composite decellularized matrix membrane was observed by naked eyes, and whether liquid seeps or not on an outer surface of the spherical composite decellularized matrix membrane was wiped by hands shown in FIG. 4; and

meanwhile, another composite decellularized matrix membrane was taken, a non-testing end was blocked, artificial urine at a temperature of 37+/−2° C. was filled in the spherical composite decellularized matrix membrane, a pressure value was applied to a testing end and at least kept for 12 h, whether the sample leaks or not was visually inspected, and test results were shown in the following table.

| | | | Comparative | Comparative |
|---|---|---|---|---|
| | Example 6 | Example 7 | example 1 | example 2 |
| Normal | No water | No water | No water | No water |
| pressure | infiltration | infiltration | infiltration | infiltration |
| Pressurized | No leakage | No leakage | No leakage | No leakage |
| for 2 Kpa | | | | |
| Pressurized | No leakage | No leakage | Slight | Slight |
| for 4 Kpa | | | leakage | leakage |
| Pressurized | No leakage | No leakage | Large | Large |
| for 6 Kpa | | | amount | amount |
| | | | of leakage | of leakage |
| Pressurized | No leakage | No leakage | Rupture | Rupture |
| for 8 Kpa | | | | |

Testing results: A water infiltration-preventing performance of the membrane in examples 6 and 7 was better than that of the membrane in comparative examples 1 and 2. Besides, the membrane in examples 6 and 7 can bear a positive pressure pressurization of 8 KPa at most, while a critical value capable of being borne by a human bladder was 40 cm of a water column, namely about 4 KPa. The water infiltration-preventing performance of the membrane in examples 6 and 7 can reach a standard of the human bladder.

### Absorbable Degradation Experiment

Test samples: composite decellularized matrix membrane prepared in example 5.1.

### Test Method:

Rabbit bladder surgery: (1) anesthesia: an experimental rabbit was anesthetized by an intraperitoneal injection with sodium pentobarbital (40 mg/kg) at a concentration of 1%.

(2) Skin preparation: hairs on an abdomen of the experimental rabbit were removed in a sterile environment and the skin was cleaned and disinfected with 2% iodophor.

(3) A wound with a length of 7-8 cm was cut on the abdominal skin of the experimental rabbit, a bladder was found out, a wound with a length of 1 cm was cut on the bladder, and a sample was cut to a proper size and attached to the wound to be sutured.

(4) Activities and feeding of the experimental rabbit were observed periodically and the experimental rabbit was dissected 1 month after the surgery for a degradation study.

Porcine bladder surgery: (1) 30 min before a surgery, a pig was intramuscularly injected with 0.05 mg/kg of atropine, 0.1 mg/kg of midazolam and 5 mg of a morphine hydrochloride injection, the pig was in a supine position with four limbs fixed, an ear vein was punctured, and 5% of a glucose injection was dripped into an abdomen at a lower abdominal center incision.

(2) An incision was 15-20 cm long. Skin at two sides of the incision was fixed by using a thumb and a forefinger of a surgeon, a tip of a scalpel was vertically punctured into the skin, then the scalpel was rotated to a 45° oblique angle of a skin surface, the skin and a tissue 5 cm below the skin were uniformly incised by using the scalpel, and the scalpel was rotated to be 90° to a vertical direction to a skin surface and taken out; if a cut length of a subcutaneous tissue was shorter than that of the skin, the subcutaneous tissue can be cut by scissors; and a cutting force should be suitable to cut out the skin at one time, such that an incision edge was neat and linear, and uneven edges caused by multiple times of incision to influence healing were avoided.

(3) Subcutaneous fat, muscle, and fascia were isolated. The sample was clamped by two hemostatic forceps and lifted upwards by a surgeon and cut between the two forceps. A proper force was exerted. A tip of the scalpel was always upward to avoid injuring deep organs and tissues. Abdominal wall muscles at 0.5 cm away from left and right sides of a center line of an abdominal wall were respectively clamped by using toothed forceps respectively, the scalpel was used vertically to cut a small opening, and the abdominal wall muscles were cut along the center line.

(4) Sterile gauze was soaked by normal saline and then spread near the incision, such that a subsequent operation was convenient, a bladder was reached, and a wound of 2-3 cm of the bladder was longitudinally cut.

(5) Prepared bladder support materials were respectively sewn on the experimental animal, whether the materials were firm or not was checked, a 0-4 micro suture was used, continuous suture and discontinuous overlock were performed, retention catheterization was performed after a repair, and a water injection test was performed.

(6) Continuous suture was performed layer by layer with 2-0 silk thread and abdominal wall muscles and skin were closed. Finally, skin incision was adjusted by using toothed forceps for folding and the skin was sterilized by iodophor for 2 times.

(7) An inhalation of an anesthetic was stopped after the surgery was finished, the experimental animal was given a full oxygen breathing, and after the experimental animal was awake, a tracheal catheter was removed after the experimental animal recovered a spontaneous breathing.

(8) Activities and feeding of the experimental pig were observed periodically and the experimental pig was dissected 2 month after the surgery for a degradation study.

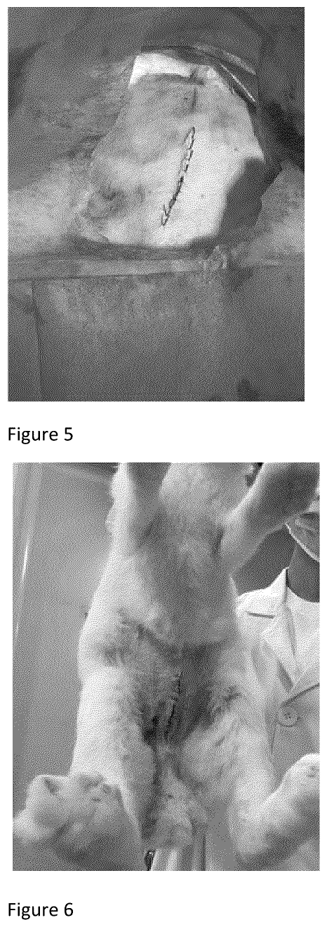

Testing results: a condition of the experimental rabbit 1 month after the surgery was shown in FIG. 6. The experimental rabbit had a good food intake and survival, and normal urination. A suture condition of the experimental rabbit 1 month after the surgery was shown in FIG. 5, which showed that the wound recovered well without infiltration and red swollen, and the surgery was good.

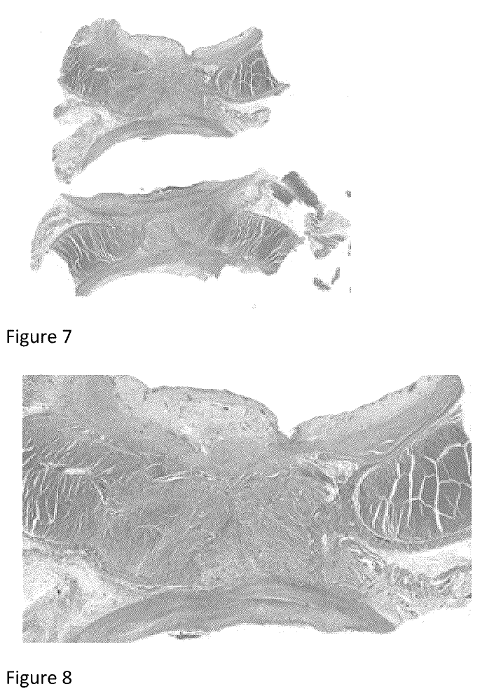

Pathological figures of the bladder of the experimental rabbit were shown in FIGS. 7 and 8. No bladder cell gaps can be seen at radians of pathological sections in figures, which indicated that a repaired part of the bladder was well healed, the bladder support material was degraded, and bladder cells crawled well.

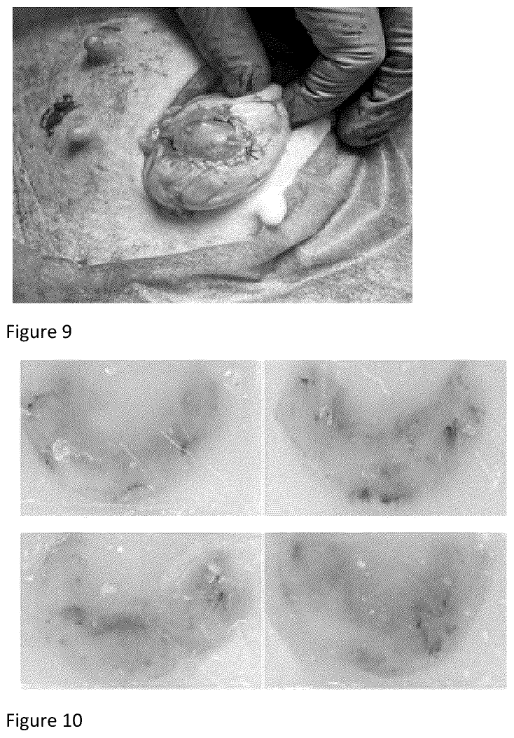

A suture condition of the experimental pig was shown in FIG. 9, which showed a completion of an open bladder repair in the pig.

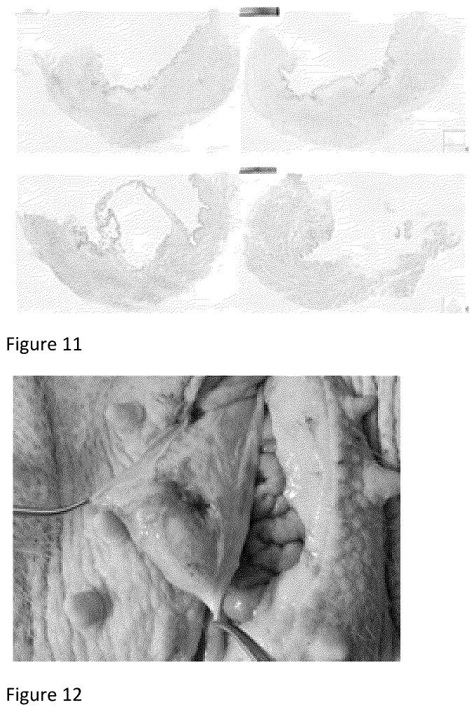

Pathological sections of specimens taken 2 months after the surgery were shown in FIGS. 10 and 11. Bladder cells in the pathological sections crawled well, which indicated that healing of a bladder tissue was good, a bladder support was dissolved, and the bladder tissue basically met morphological requirements of an original tissue.

In conclusion, the rabbit bladder surgery and the porcine bladder surgery both showed that the decellularized matrix membrane prepared by the present disclosure can prevent water infiltration and was absorbably degradable when used for a bladder repair and a total bladder repair. Predictably, in a clinical practice, when the composite decellularized matrix membrane provided by the present disclosure can be degraded, absorbed, and free of residue, a healed bladder was mainly an autologous tissue.

### Example 8 Preparation of Composite Decellularized Matrix Membrane (PU Polymer Membrane)

A preparation method of the composite decellularized matrix membrane provided by the present example comprised the following steps:
(1) 1 g of polyurethane (PU) was weighed and added to 100 ml of acetone, and the mixture was wrapped in tin paper to avoid light and stirred at a room temperature for 60 min until completely dissolved to obtain a polymer solution;
(2) a cut decellularized matrix membrane (4 cm×4 cm) of example 1.1 was completely soaked with deionized water, laid, and put still in a ventilated place until water on a surface was evaporated; 2 mL of the polymer solution was taken and coated on the surface of the decellularized matrix membrane as to obtain the decellularized matrix membrane with a polymer membrane after the solvent acetone was evaporated; and
(3) another piece of cut decellularized matrix membrane was completely soaked with deionized water, laid on the decellularized matrix membrane with the polymer membrane prepared in step (2), and put still for 60 min to obtain a composite decellularized matrix membrane.

### Example 9 Preparation of Composite Decellularized Matrix Membrane (PLGA Polymer Membrane)

The preparation steps and the amount of the polymer of the example were basically the same as those in example 8 except that polylactic acid-glycolic acid was selected as a polymer material and chloroform was selected as a solvent.

### Example 10 Preparation of Composite Decellularized Matrix Membrane (PDMS Polymer Membrane)

The preparation steps and the amount of the polymer of the example were basically the same as those in example 8 except that polydimethylsiloxane was selected as a polymer material and chloroform was selected as a solvent.

### Example 11 Preparation of Composite Decellularized Matrix Membrane (PVC Polymer Membrane)

The preparation steps and the amount of the polymer of the example were basically the same as those in example 8 except that polyvinyl chloride was selected as a polymer material and cyclohexanone was selected as a solvent.

### Example 12 Preparation of Composite Decellularized Matrix Membrane (PVA Polymer Membrane)

The preparation steps and the amount of the polymer of the example were basically the same as those in example 8 except that polyvinyl alcohol was selected as a polymer material and chloroform was selected as a solvent.

### Example 13 Preparation of Composite Decellularized Matrix Membrane (PHA Polymer Membrane)

The preparation steps and the amount of the polymer of the example were basically the same as those in example 8 except that polyhydroxy fatty acid ester was selected as a polymer material and chloroform was selected as a solvent.

### Example 14 Preparation of Composite Decellularized Matrix Membrane (PEEK Polymer Membrane)

The preparation steps and the amount of the polymer of the example were basically the same as those in example 8 except that polyether-ether-ketone was selected as a polymer material and chloroform was selected as a solvent.

### Example 15 Preparation of Composite Decellularized Matrix Membrane (PAN Polymer Membrane)

The preparation steps and the amount of the polymer of the example were basically the same as those in example 8 except that polyacrylonitrile was selected as a polymer material.

### Example 16 Preparation of Composite Decellularized Matrix Membrane (PS Polymer Membrane)

The preparation steps and the amount of the polymer of the example were basically the same as those in example 8 except that polystyrene was selected as a polymer material and dimethyl sulfoxide was selected as a solvent.

### Anti-Inflammatory Animal Experiment of Bladder

Test samples: the composite decellularized matrix membranes prepared in examples 5.1 and 8-16.

Test method: the test method was the same as the porcine bladder surgery.

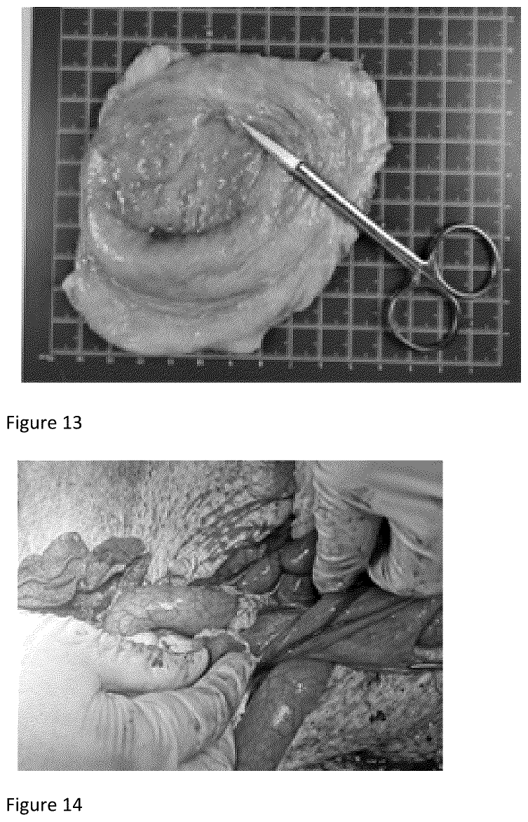

Test results: postoperative conditions of the composite decellularized matrix membranes prepared from SIS with PU, PCL, PLGA, PDMS, PVA, and PHA polymer materials were shown in FIGS. 12 and 13. FIG. 12 showed a surgery of example 5.1 (PCL) in an anti-inflammatory animal experiment of a bladder. FIG. 13 showed a surgery of example 8 (PU) in an anti-inflammatory animal experiment of a bladder. From the figures, it can be seen that a wound was free of infiltration and red swollen, which indicates that no inflammation occurred in the animal experiment. Other materials had the same results as PU. No infiltration and red swollen were shown.

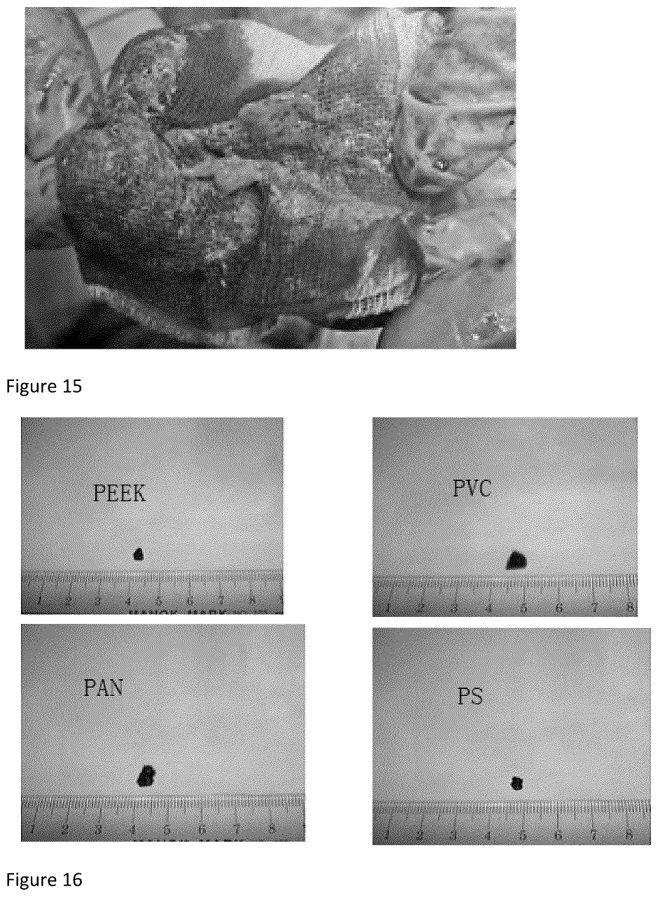

Postoperative conditions of the composite decellularized matrix membranes prepared from SIS with PVC, PEEK, PAN, and PS polymer materials were shown in FIGS. 14 and 15, wherein FIG. 14 showed a surgery of example 11 (PVC) in an anti-inflammatory animal experiment of a bladder and FIG. 15 showed a surgery of example 15 (PAN) in an anti-inflammatory animal experiment of a bladder. From the figures, it can be seen that an animal wound was free of red swollen and infiltration, which indicated that mild or moderate inflammation occurred in the animal experiment. Therefore, the composite decellularized matrix membranes prepared from SIS with PU, PCL, PLGA, PDMS, PVA, and PHA polymer materials can achieve an anti-inflammatory effect.

### Artificial Bladder Cell Experiment

### Experiment 1

Test samples: the composite decellularized matrix membrane prepared in example 5.1.

Test method: normal urinary tract epithelial cells SVHUC-1 transfected with a GFP fluorescent protein were inoculated on a bladder patch repaired by using the composite decellularized matrix membranes prepared in example 5.1, after the cells were adhered to a wall for 8 h, the cells were fully rinsed for 3 times, then adherent cells were desorbed, and a fluorescence intensity was measured.

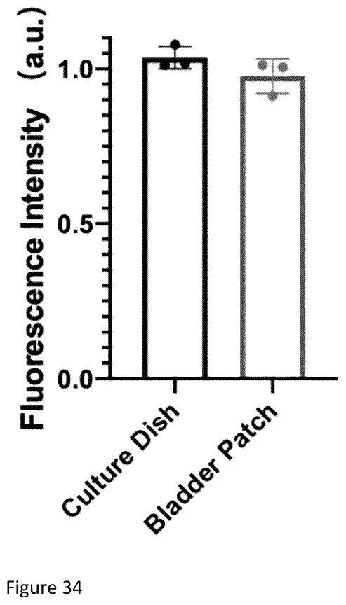

Testing results: as shown in FIG. 34, it was demonstrated that the composite decellularized matrix membrane of the present disclosure had an excellent cell adhesion affinity. Similarly, the cell adhesion affinity of PU, PLGA, PDMS, PVA, and PHA was also tested to be the same as that of PCL without significant difference. But the composite decellularized matrix membranes prepared from the PVC, PEEK, PAN, and PS polymer materials showed a relatively week cell adhesion affinity (without significant difference) and had significant differences compared with the composite membranes prepared from PCL, PU, PLGA, PDMS, PVA, and PHA (P less than 0.05). Specific experimental data was omitted.

### Experiment 2

Test samples: the composite decellularized matrix membrane prepared in example 5.1.

Test method: a medium cultured with a bladder patch repaired using the composite decellularized matrix membrane prepared in example 5.1 for a certain period of time (day 1, day 3, day 6, and day 9) was cultured together with normal urinary tract epithelial cells SVHUC-1 transfected with a GFP fluorescent protein for 24 h. A cell activity was examined.

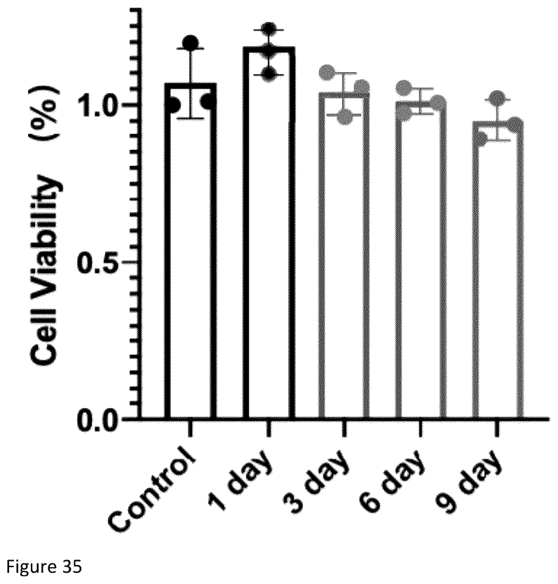

Test results: the cell activity was shown in FIG. 35, which showed that the composite decellularized matrix membrane of the present disclosure had very good cell compatibility. Similarly, the cell compatibility of PU, PLGA, PDMS, PVA, and PHA was also tested to be the same as that of PCL without significant difference. But the composite decellularized matrix membranes prepared from the PVC, PEEK, PAN, and PS polymer materials showed a relatively week cell compatibility (without significant difference) and had significant differences compared with the composite membranes prepared from PCL, PU, PLGA, PDMS, PVA, and PHA (P less than 0.05). Specific experimental data was omitted.

### Calculus-Preventing Experiment

Test samples: the composite decellularized matrix membranes prepared in examples 5.1 and 8-16.

Test method: the test method was the same as the porcine bladder surgery.

Test results: calculus formation of the composite decellularized matrix membranes prepared from SIS with PVC, PEEK, PAN, and PS polymer materials was shown in FIG. 16. It can be seen from the figure that SIS and PVC had formation of a calculus of 6 mm, SIS and PEEK had formation of a calculus of 5 mm, SIS and PAN had formation of a calculus of 7 mm, and SIS and PS had formation of a calculus of 5 mm. The composite decellularized matrix membranes prepared from SIS with PU, PCL, PLGA, PDMS, PVA, and PHA polymer materials do not form calculi when being used for the bladder surgery.

### Toughness Test

Test samples: the composite decellularized matrix membranes prepared in examples 5.1 and 8-16.

Test method: the composite decellularized matrix membrane was folded at 180° or folded again.

Test results: a qualified toughness standard was that an artificial bladder was folded at 180° in any direction and then folded at 180° again in the same direction, an artificial bladder should be recovered, and defects such as folding or rupture and the like should not occur in an artificial bladder body by visual inspection.

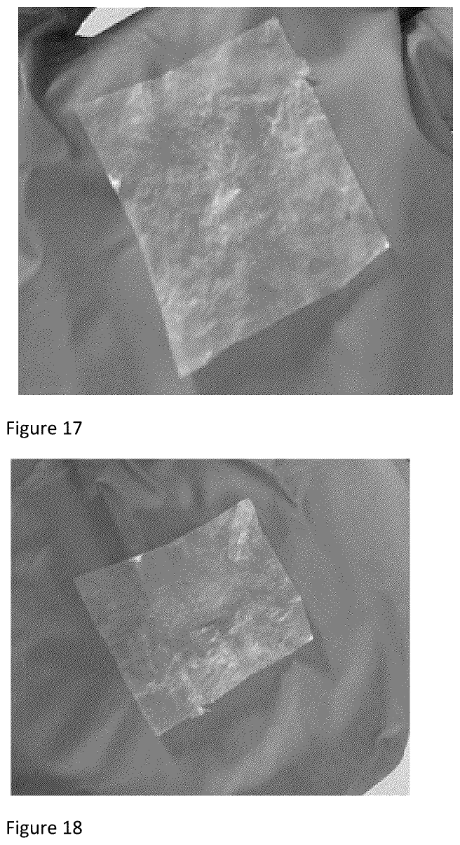

The test results of the composite decellularized matrix membranes prepared from SIS with PU, PCL, PLGA, PDMS, PEEK, and PS polymer materials were shown in FIGS. 17 and 18, wherein FIG. 17 showed a test result of the composite decellularized matrix membrane prepared from the SIS and PCL polymer materials and FIG. 18 showed a test result of the composite decellularized matrix membrane prepared from the SIS and PU polymer materials. From the figures, it can be seen that after folded at 180° in any direction and then folded at 180° again in the same direction, the artificial bladder recovered and defects such as folding or rupture and the like did not occur by visual inspection.

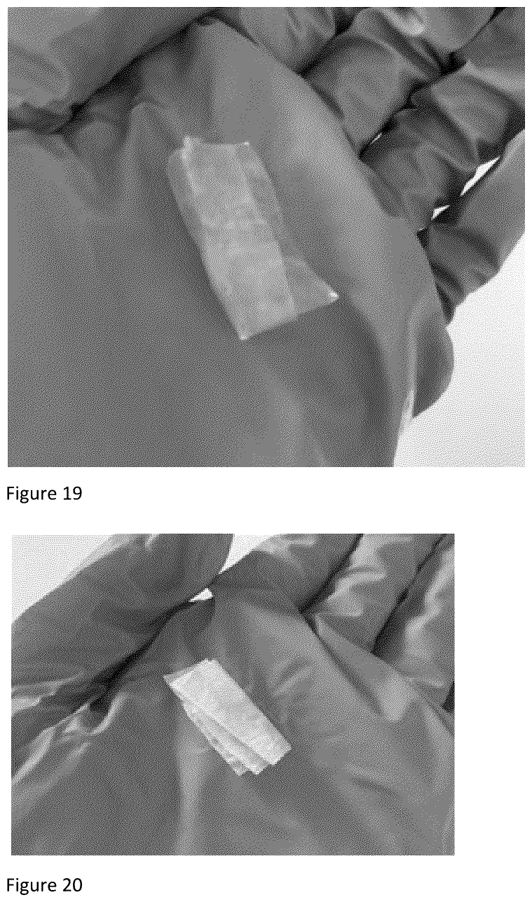

The test results of the composite decellularized matrix membranes prepared from SIS with PVC and PHA polymer materials were shown in FIGS. 19 and 20, wherein FIG. 19 showed a test result of the composite decellularized matrix membrane prepared from the SIS and PVC polymer materials and FIG. 20 showed a test result of the composite decellularized matrix membrane prepared from the SIS and PHA polymer materials. From the figures, it can be seen that after folded at 180° and then folded at 180° again, the artificial bladder did not recover but defects such as folding or rupture and the like did not occur by visual inspection.

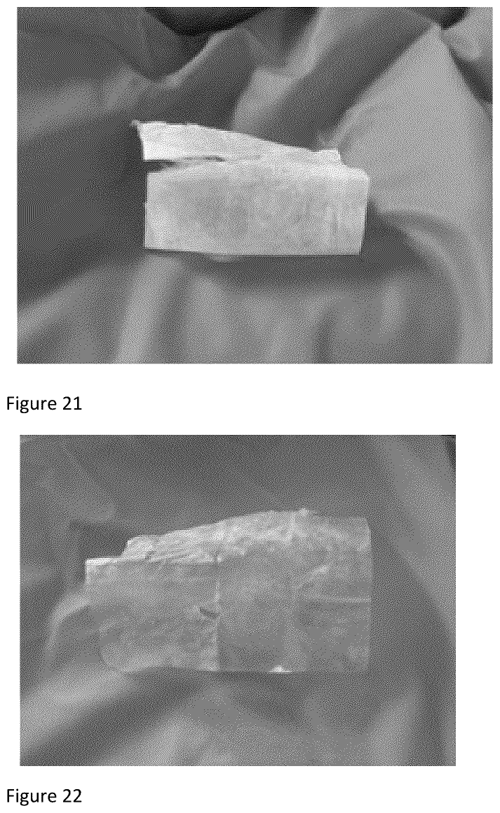

The test results of the composite decellularized matrix membranes prepared from SIS with PVA and PAN polymer materials were shown in FIGS. 21 and 22, wherein FIG. 21 showed a test result of the composite decellularized matrix membrane prepared from the SIS and PVA polymer materials and FIG. 22 showed a test result of the composite decellularized matrix membrane prepared from the SIS and PAN polymer materials. From the figures, it can be seen that after folded at 180° and then folded at 180° again, the artificial bladder did not recover and defects such as folding or rupture and the like occurred by visual inspection. Therefore, the composite decellularized matrix membranes prepared from SIS with PU, PCL, PLGA, PDMS, PEEK, and PS polymer materials had a good toughness.

The above experimental results were specifically shown in the following table:

| | Example 5.1 | Example 8 | Examples 9 | Example 10 | Example 11 |
|---|---|---|---|---|---|
| | (PCL) | (PU) | (PLGA) | (PDMS) | (PVC) |
| Inflammatory | No | No | No | No | Slight |
| condition | inflammation | inflammation | inflammation | inflammation | inflammation |
| Formation | No | No | No | No | 6 mm |
| of calculi | calculi | calculi | calculi | calculi | calculi can be seen. |
| Toughness | After folded | After folded | After folded | After folded | After folded |
| | at 180° in any | at 180° in any | at 180° in any | at 180° in any | at 180° and |
| | direction and | direction and | direction and | direction and | then folded at 180° |
| | then folded at 180° | then folded at 180° | then folded at 180° | then folded at 180° | again, the |
| | again in the same | again in the same | again in the same | again in the same | artificial bladder |
| | direction, an | direction, an | direction, an | direction, an | did not recover but |
| | artificial bladder | artificial bladder | artificial bladder | artificial bladder | defects such as |
| | recovered and | recovered and | recovered and | recovered and | folding or rupture |
| | defects such as | defects such as | defects such as | defects such as | and the like did |
| | folding or rupture | folding or rupture | folding or rupture | folding or rupture | not occur by visual |
| | and the like did | and the like did | and the like did | and the like did | inspection. |
| | not occur by visual | not occur by visual | not occur by visual | not occur by visual | |
| | inspection. | inspection. | inspection. | inspection. | |
| | Examples 12 | Example 13 | Example 14 | Example 15 | Example 16 |
| | (PVA) | (PHA) | (PEEK) | (PAN) | (PS) |
| Inflammatory | No | No | Slight | Moderate | Slight |
| condition | inflammation | inflammation | inflammation | inflammation | inflammation |
| Formation | No | No | 5 mm | 7 mm | 5 mm |
| of calculi | calculi | calculi | calculi can be seen. | calculi can be seen. | calculi can be seen. |
| Toughness | After folded | After folded | After folded | After folded | After folded |
| | at 180° and | at 180° and | at 180° in any | at 180° and | at 180° in any |
| | then folded at 180° | then folded at 180° | direction and | then folded at 180° | direction and |
| | again, the | again, the | then folded at 180° | again, the | then folded at 180° |
| | artificial bladder | artificial bladder | again in the same | artificial bladder | again in the same |
| | did not recover and | did not recover but | direction, an | did not recover and | direction, an |
| | defects such as | defects such as | artificial bladder | defects such as | artificial bladder |
| | folding or rupture | folding or rupture | recovered and | folding or rupture | recovered and |
| | and the like | and the like did | defects such as | and the like | defects such as |
| | occurred by visual | not occur by visual | folding or rupture | occurred by visual | folding or rupture |
| | inspection. | inspection. | and the like did | inspection. | and the like did |
| | | | not occur by visual | | not occur by visual |
| | | | inspection. | | inspection. |

Result analysis: when the composite decellularized matrix membranes prepared from the decellularized matrix membrane and PCL, PU, PLGA, and PDMS used for a bladder patch, inflammation and calculus formation did not occur. Meanwhile, after folded at 180° in any direction and then folded at 180° again in the same direction, the artificial bladder can restore to an original shape and it was determined that defects such as folding or rupture and the like did not occur according to a folded appearance. An inflammation phenomenon and calculus formation did not occur, indicating that the artificial bladder prepared from the composite decellularized matrix membrane provided by the present disclosure in clinical practice did not enable a patient to be like a patient subjected to a sigmoid colorectal cystectomy who easily suffered from serious complications such as urinary tract inflammation, water electrolyte acid-base equilibrium disorder, even intestinal tumors and the like. The artificial bladder had few complications, did not damage an intestinal tract, and greatly reduced surgical risks and patient injury. A qualified toughness indicated that after the composite decellularized matrix membrane provided by the present disclosure was used for the bladder surgery, the membrane had a lower distortion resistance and good deformation performance in a human body, no folding and rupture fracture occurred due to deformation of the composite membrane, urine leakage was prevented, patch suturing was not affected during bladder contracture, foreign body sensation did not easily occur after the surgery, and the membrane had good stability.

### Example 17 Preparation of Tubular Composite Decellularized Matrix Membrane (PCL Polymer Membrane)

A preparation method of the tubular composite decellularized matrix membrane provided by the present example comprised the following steps:
(1) preparation of PCL solution: 1 g of polycaprolactone (PCL) was weighed and added to 100 ml of chloroform, and the mixture was wrapped in tin paper to avoid light and stirred at a room temperature for 1 h until completely dissolved;
(2) a layer of a plastic film was wrapped on a surface of a glass rod with a diameter of 5 mm;
(3) the decellularized matrix membrane of example 1.1 was completely soaked, wound on a surface of the glass rod with a diameter of 5 mm, and placed in a ventilated place until water on a surface was evaporated;
(4) 0.5 mL of the PCL solution was taken and coated on the surface of the decellularized matrix membrane until the solvent chloroform was completely volatilized; and
(5) then another piece of the completely soaked decellularized matrix membrane with an appropriate size was taken and carefully laid on the decellularized matrix membrane with a polymer membrane manufactured in step (4) until the membrane was completely dried to obtain a tubular composite decellularized matrix membrane shown in FIG. 33.

### Example 18 Preparation of Tubular Composite Decellularized Matrix Membrane (PACA Polymer Membrane)

The preparation steps of the example were basically the same as those in example 17 except that polyalkylcyanoacrylate (PACA) was selected as a polymer material.

### Example 19 Preparation of Tubular Composite Decellularized Matrix Membrane (PLGA Polymer Membrane)

The preparation steps of the example were basically the same as those in example 17 except that polylactic acid-glycolic acid (PLGA) was selected as a polymer material.

### Example 20 Preparation of Tubular Composite Decellularized Matrix Membrane (PLLA Polymer Membrane)

The preparation steps of the example were basically the same as those in example 17 except that L-polylactic acid (PLLA) was selected as a polymer material.

### Example 21 Preparation of Tubular Composite Decellularized Matrix Membrane (PE Polymer Membrane)

The preparation steps of the example were basically the same as those in example 17 except that polyethylene was selected as a polymer material.

### Example 22 Preparation of Tubular Composite Decellularized Matrix Membrane (PTFE Polymer Membrane)

The preparation steps of the example were basically the same as those in example 17 except that polytetrafluoroethylene (PTFE) was selected as a polymer material.

### Example 23 Preparation of Tubular Composite Decellularized Matrix Membrane (PP Polymer Membrane)

The preparation steps of the example were basically the same as those in example 17 except that polypropylene (PP) was selected as a polymer material.

### Example 24 Preparation of Tubular Composite Decellularized Matrix Membrane (PC Polymer Membrane)

The preparation steps of the example were basically the same as those in example 17 except that polycarbonate (PC) was selected as a polymer material.

### Example 25 Preparation of Tubular Composite Decellularized Matrix Membrane (PVP Polymer Membrane)

The preparation steps of the example were basically the same as those in example 17 except that polyvinylpyrrolidone (PVP) was selected as a polymer material.

### Example 26 Preparation of Tubular Composite Decellularized Matrix Membrane (PNVP Polymer Membrane)

The preparation steps of the example were basically the same as those in example 17 except that poly(N-vinylpyrrolidone) (PNVP) was selected as a polymer material.

### Anti-Inflammatory Animal Experiment of Ureter

Test samples: composite decellularized matrix membranes prepared in examples 17-26.

Test method: the test method was the same as the porcine bladder surgery. A test part was replaced into a ureter.

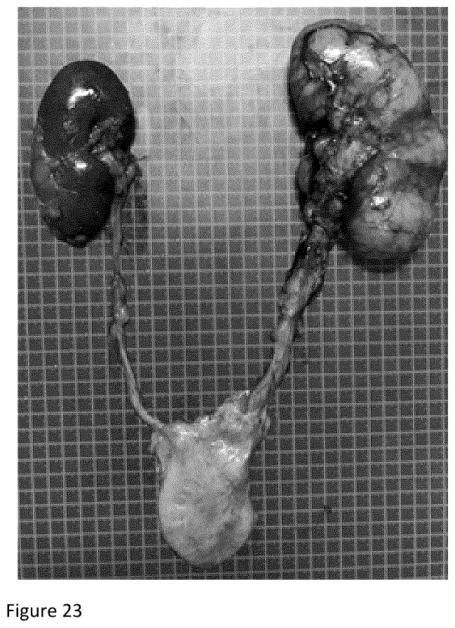

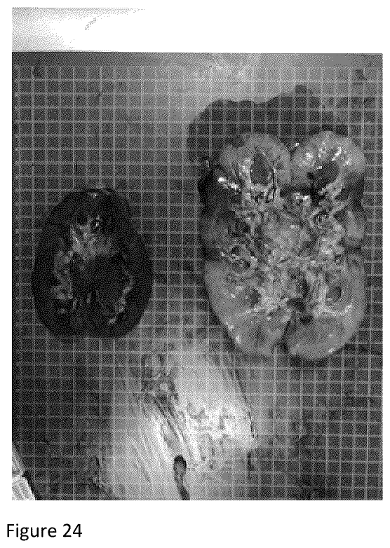

Test results: the test results of the composite decellularized matrix membranes prepared from SIS with PACA, PCL, PLGA, PLLA, and PTEF polymer materials were shown in FIGS. 23-24, wherein FIG. 23 showed a surgical condition of the composite decellularized matrix membrane prepared from the SIS and PCL polymer materials and FIG. 24 showed a surgical condition of the composite decellularized matrix membrane prepared from the SIS and PLGA polymer materials. From FIGS. 23-24, it can be seen that a ureter was free of infiltration and red swollen, indicated that the ureter was free of inflammation.

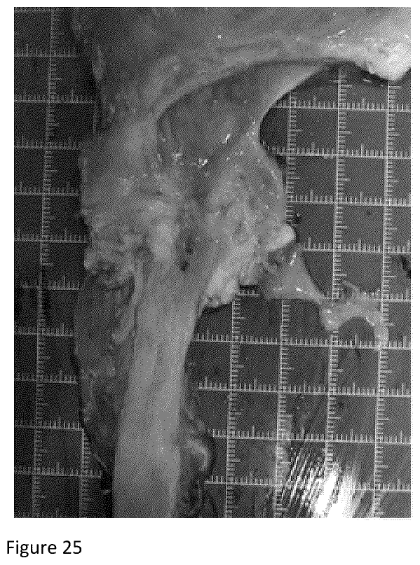

A postoperative condition of the composite decellularized matrix membranes prepared from SIS with PE, PP, PC, PVP, and PNVP polymer materials were shown in FIG. 25. FIG. 25 showed a surgical condition of the composite decellularized matrix membrane prepared from SIS and PP polymer materials. From the figure, it can be seen that a infiltration phenomenon occurred, indicating mild or moderate inflammation.

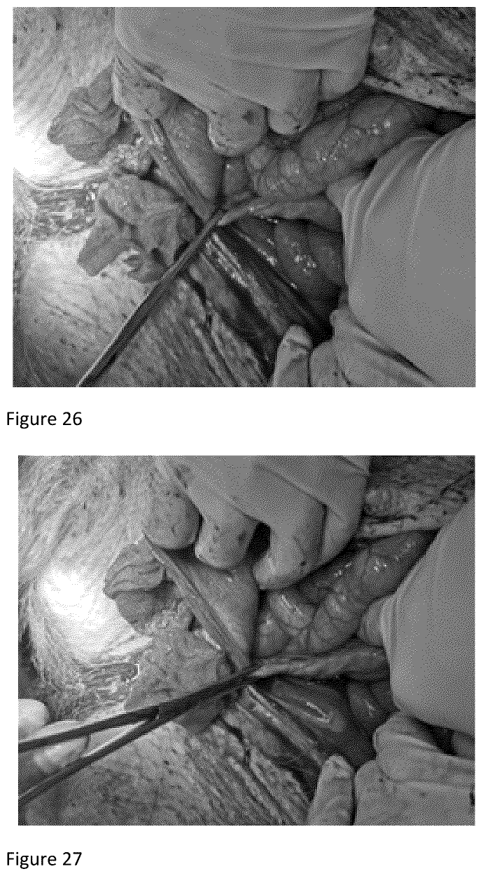

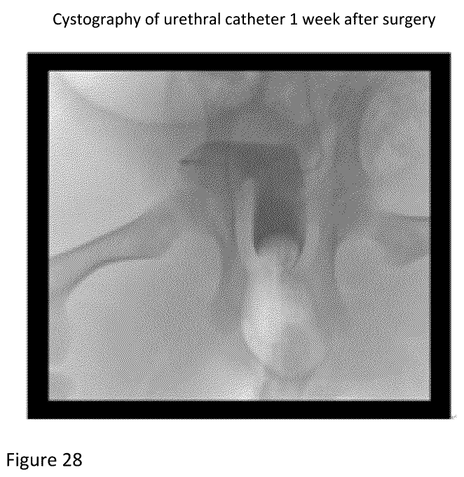

Results of a postoperative stenosis test of the composite decellularized matrix membranes prepared from SIS with PACA, PCL, PLGA, PLLA, PTEF, PP, and PNVP polymer materials were shown in FIG. 26 and FIG. 28. FIG. 26 showed a surgical condition of the composite decellularized matrix membrane prepared from SIS and PCL polymer materials and FIG. 28 showed a cystography of a urethral catheter 1 week after a surgery of the composite decellularized matrix membrane prepared from SIS and PCL polymer materials. FIG. 26 showed that a pipeline of the ureter was unobstructed. FIG. 28 showed that the pipeline of the ureter was unobstructed, which indicated that the ureter did not have a stenosis. Test results of the composite decellularized matrix membranes prepared from SIS with PE, PC, and PVP polymer materials were shown in FIG. 27. FIG. 27 showed a surgical condition of the composite decellularized matrix membrane prepared from SIS and PE polymer materials. FIG. 27 showed that the pipeline of the ureter was obstructed and had a relatively small or moderate stenosis.

### Rigidity and Strength Experiment

Test samples: composite decellularized matrix membranes prepared in examples 17-26.

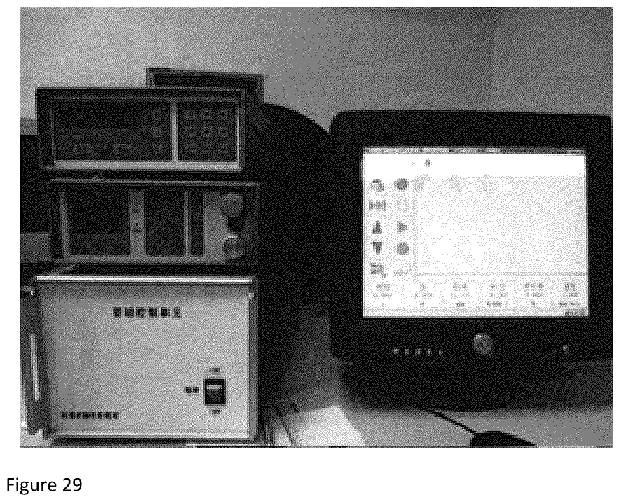

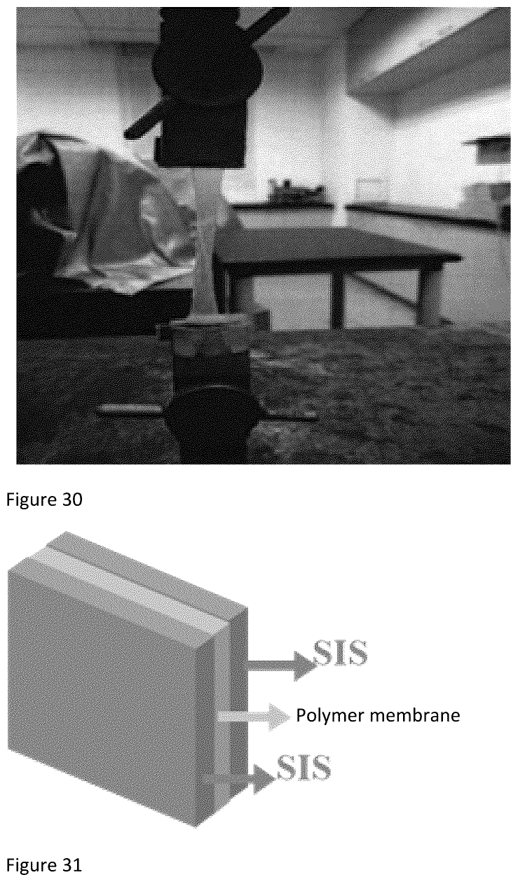

Test method: a test device shown in FIGS. 29-30 was used for testing.

Test results: acceptance criteria were that when a force of 1 N was applied on a product, a deflection should be less than or equal to 25 mm and a deformation lasting for 1 min should be less than or equal to 2.5 mm. Experimental results of a ureter were shown in the following table:

| | Example 17 | Example 18 | Examples 19 | Example 20 | Example 21 |
|---|---|---|---|---|---|
| | (PCL) | (PACA) | (PLGA) | (PLLA) | PE |
| Inflammatory | No | No | No | No | Moderate |
| condition | inflammation | inflammation | inflammation | inflammation | inflammation |
| Stenosis | No | No | No | No | Relatively |
| condition | stenosis | stenosis | stenosis | stenosis | small stenosis |
| Rigidity | When a force of | When a force of | When a force of | When a force of | When a force of |
| and | 1N is applied | 1N is applied | 1N is applied | 1N is applied | 1N is applied |
| strength | on a product, a | on a product, a | on a product, a | on a product, a | on a product, a |
| | deflection | deflection | deflection | deflection | deflection |
| | is 16 mm and a | is 15 mm and a | is 18 mm and a | is 20 mm and a | is 22 mm and a |
| | deformation | deformation | deformation | deformation | deformation |
| | lasting for 1 min | lasting for 1 min | lasting for 1 min | lasting for 1 min | lasting for 1 min |
| | is 1.4 mm. | is 1.9 mm. | is 1.5 mm. | is 1.2 mm. | is 2.5 mm. |
| | Example 22 | Example 23 | Example 24 | Example 25 | Example 26 |
| | PTFE | PP | PC | PVP | PNVP |
| Inflammatory | No | Slight | Slight | Moderate | Slight |
| condition | inflammation | inflammation | inflammation | inflammation | inflammation |
| Stenosis | No | No | Relatively | Moderate | No |
| condition | stenosis | stenosis | small stenosis | stenosis | stenosis |
| Rigidity | When a force of | When a force of | When a force of | When a force of | When a force of |
| and | 1N is applied | 1N is applied | 1N is applied | 1N is applied | 1N is applied |
| strength | on a product, a | on a product, a | on a product, a | on a product, a | on a product, a |
| | deflection | deflection | deflection | deflection | deflection |
| | is 28 mm and a | is 26 mm and a | is 29 mm and a | is 18 mm and a | is 22 mm and a |
| | deformation | deformation | deformation | deformation | deformation |
| | lasting for 1 min | lasting for 1 min | lasting for 1 min | lasting for 1 min | lasting for 1 min |
| | is 2.7 mm. | is 2.2 mm. | is 2.3 mm. | is 2.3 mm. | is 1.8 mm. |

Result analysis: when the composite decellularized matrix membranes prepared from SIS with PCL, PACA, PLGA, and PLLA polymer materials used in a clinical experiment of the ureter, inflammation and stenosis did not occur, rejection reaction brought by an allogeneic tissue and ureteral hydrops and hydronephrosis caused by postoperative stenosis were avoided, and a problem of histocompatibility easily caused by an artificial material replacement was solved. Occurrences of complications of a patient were avoided, and surgical risks and pains of the patient were greatly relieved. Meanwhile, rigidity and strength of the membrane enable the membrane to have a certain supporting force in a body of the patient. In a normal floating range of the ureter, no contracture occurred and perforation or displacement deformation was avoided.

All the patents and publications mentioned in the description of the present disclosure indicate that these are public technologies in the art and can be used by the present disclosure. All the patents and publications cited herein are listed in the references, just as each publication is specifically referenced separately. The present disclosure described herein can be realized in the absence of any one element or multiple elements, one restriction or multiple restrictions, where the limitation is not specifically described here. For example, in each example, the terms “comprise”, “substantially composed of” and “composed of” can be replaced by the remaining two terms of either. The so-called “a” here only means “a kind”, not excluding only one, but also can indicate two or more. The terms and expressions used herein are descriptive, without limitation. Besides, there is no intention to indicate that these terms and interpretations described in the description exclude any equivalent features. However, it can be known that any appropriate changes or modifications can be made within the scope of the present disclosure and claims. It can be understood that the examples described in the present disclosure are some preferred examples and features. A person skilled in the art can make some modifications and changes according to the essence of the description of the present disclosure. These modifications and changes are also considered to fall within the scope of the present disclosure and the scope limited by independent claims and dependent claims.

1. A composite decellularized matrix membrane, comprising a decellularized matrix membrane and a polymer membrane, wherein the composite decellularized matrix membrane comprises two layers of the decellularized matrix membranes and one layer of the polymer membrane, and the one layer of the polymer membrane is located between the two layers of the decellularized matrix membranes.

2. The composite membrane according to claim 1, wherein the decellularized matrix membrane is prepared from a biomaterial of a porcine small intestine, a porcine bladder, and porcine skin.

3. The composite membrane according to claim 2, wherein the polymer membrane is prepared from a polymer material of polycaprolactone, polylactic acid, and polyurethane.

4. The composite membrane according to claim 2, wherein the decellularized matrix membrane is prepared from a submucosa of a porcine small intestine.

5. The composite membrane according to claim 4, wherein the polymer membrane is prepared from polycaprolactone and the composite membrane is usable for preparing an absorbably degradable and good-toughness artificial bladder capable of preventing calculi, water infiltration, and inflammation.

6. The composite membrane according to claim 4, wherein the polymer membrane is prepared from polylactic acid and the composite membrane is usable for preparing an absorbably degradable artificial bladder capable of preventing calculi and water infiltration.

7. The composite membrane according to claim 4, wherein the polymer membrane is prepared from polyurethane.

8. The composite membrane according to claim 4, wherein the polymer membrane is prepared from at least one polymer material of polydimethylsiloxane, polylactic acid-glycolic acid, polyvinyl alcohol, and polyhydroxy fatty acid ester, and wherein the composite membrane is usable for preparing an artificial bladder with a good toughness.

9. The composite membrane according to claim 4, wherein the polymer membrane is prepared from at least one polymer material of polydimethylsiloxane, polylactic acid-glycolic acid, polyvinyl alcohol, and polyhydroxy fatty acid ester, and wherein the composite membrane is usable for preparing an artificial bladder capable of preventing inflammation.

10. The composite membrane according to claim 4, wherein the decellularized matrix membrane is prepared by the following steps:
(1) physically treating a porcine small intestine and removing an adhesive substance of a submucosa to obtain the submucosa;
(2) immersing the obtained submucosa of the porcine small intestine in a decellularizing solution (adding 10 mL of Triton and 1 mL of ammonium hydroxide to 1,000 mL of deionized water) and placing the submucosa in a refrigerator at 4° C. for oscillating at 100 rpm for 12 h;
(3) after rinsing with a large amount of deionized water, placing the submucosa in 1 L of a degreasing solution (methanol/chloroform=1:1) for soaking for 24 h;
(4) after rinsing with deionized water, placing the submucosa in 1 L of anhydrous ethanol for oscillating and soaking for 24 h and fully extracting residual methanol/chloroform to obtain a prepared decellularized matrix membrane of the submucosa of the porcine small intestine; and
(5) finally repeatedly washing the obtained decellularized matrix membrane with deionized water and freeze-drying, sealing, and storing the decellularized matrix membrane in a refrigerator at 4° C.

11. The composite membrane according to claim 4, wherein the composite decellularized matrix membrane is prepared by the following steps:
(1) weighing a polymer material, putting the same into a solvent, and shaking the solution until completely dissolved in a dark place at a room temperature to obtain a polymer solution;
(2) after completely soaking the decellularized matrix membrane with water, laying the membrane on a surface of a spherical mold, and placing the membrane in a ventilated place until water on a surface of the membrane is evaporated;
(3) coating the polymer solution on the surface of the decellularized matrix membrane after the water on the surface is evaporated, so as to obtain the decellularized matrix membrane with the polymer membrane; and
(4) after the solvent is completely volatilized, completely soaking another decellularized matrix membrane, laying the same on the decellularized matrix membrane with the polymer membrane, and after the membrane is completely dried, taking out the mold to obtain the composite decellularized matrix membrane.

12. An artificial bladder, wherein the artificial bladder comprises a composite decellularized matrix membrane, the composite membrane comprises a decellularized matrix membrane and a polymer membrane, the composite decellularized matrix membrane comprises two layers of the decellularized matrix membranes and one layer of the polymer membrane, and the one layer of the polymer membrane is located between the two layers of the decellularized matrix membranes.

13. The artificial bladder according to claim 12, wherein the decellularized matrix membrane is prepared from at least a biomaterial of a porcine small intestine, a porcine bladder, and porcine skin.

14. The artificial bladder according to claim 13, wherein the polymer membrane is prepared from at least a polymer material of polycaprolactone, polylactic acid, and polyurethane.

15. The artificial bladder according to claim 14, wherein the decellularized matrix membrane is prepared from a submucosa of a porcine small intestine.

16. The artificial bladder according to claim 15, wherein the polymer membrane is prepared from polycaprolactone.

17. The artificial bladder according to claim 15, wherein the polymer membrane is prepared from polyurethane.

18. The artificial bladder according to claim 13, wherein the polymer membrane is prepared from at least a polymer material of polydimethylsiloxane, polyurethane, polylactic acid-glycolic acid, polyvinyl alcohol, and polyhydroxy fatty acid ester.

19. The artificial bladder according to claim 18, wherein the polymer membrane is prepared from polydimethylsiloxane.
